(19) United States
(12) Patent Application Publication
(10) Pub. No.: US 2002/0137725 A1
(43) Pub. Date: Sep. 26, 2002

(54) Use of tagatose in promoting cardiovascular health

(76) Inventor: Gilbert V. Levin, Annapolis, MD (US)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 09/811,654

(22) Filed: Mar. 20, 2001

(51) Int. Cl. A61K 31/715

(52) U.S. Cl. 514/61

(57) ABSTRACT

A method for promoting cardiovascular health of a mammal in need thereof, comprising administering to said mammal an efficacious amount of tagatose to raise the HDL level of said mammal.

### BACKGROUND OF THE INVENTION

This invention relates to the use of tagatose to improve cardiovascular health by increasing the patient's High-Density Lipoproteins (HDL) level.

Low levels of HDL are a risk factor in cardiovascular health, as HDLs serve to sweep artery clogging cholesterol from the bloodstream.

U.S. Pat. No. 5,447,917 discloses and claims a method for treating diabetes by administering to a mammal an amount of D-tagatose effective to inhibit the rise in blood sugar associated with the consumption of sugar. A pilot study was conducted at the University of Maryland to investigate the long term effects of D-tagatose in humans with type-2 diabetes. During the course of this study, there was found to be an increase in HDL-cholesterol levels in each of the subjects, both the patients and the controls, treated with the tagatose.

### SUMMARY OF THE INVENTION

In accordance with this invention, there is provided a method for promoting cardiovascular health in a mammal, especially man, in need of such treatment, which comprises administering to said mammal an efficacious amount of tagatose, i.e., D-tagatose, L-tagatose or a mixture of the two isomers, to increase the HDL level of the mammal.

### DETAILED DESCRIPTION OF THE INVENTION

The tagatose may be administered to a subject in combination with a food, beverage or taken separately in powder, crystalline or liquid form. As diluent, if needed, one may use liquid or solid carriers such as water, starch, alcohol, or other non-toxic substances. Preferably, the tagatose is administered in the weight range of 50 mg/kg body weight/day to 1,500 mg/kg body weight/day. The tagatose may be administered daily, every other day, or at other prescribed frequencies. It may be administered in combination with other medications known to be suitable for use in the treatment of cardiovascular health.

### EXAMPLE 1

### Use of Tagatose in Promoting Cardiovascular Health

A human patient is diagnosed as having a lowered HDL level than that found in a previous visit. The physician is concerned that this may forecast atherosclerosis, possibly leading to a heart attack, or to a stroke. The physician prescribes that the patient take 15 grams of tagatose with each of three meals per day. After six months of such treatment, the patient's HDL level is determined to be significantly higher than it was immediately before the treatment was commenced. The physician is satisfied with the improvement and prescribes continued 5 gram doses of tagatose per meal as a means of maintaining and improving the patient's cardiovascular health.

What is claimed is:

1. A method for promoting cardiovascular health in a mammal in need of such treatment comprising administering to said mammal an efficacious amount of tagatose to raise the HDL level in the mammal.

2. The method of claim 1 wherein the mammal is a human.

3. The method of claim 1 wherein from 50 to 1,500 mg/kg body weight/day is administered to said mammal.

4. The method of claim 1 wherein the prescribed dose is taken every day.

5. The method of claim 1 wherein the prescribed dose is taken every other day.

6. The method of claim 1 wherein said tagatose is used in combination with a medication known to be useful in promoting cardiovascular health.

7. The method of claim 1 wherein the tagatose is D-tagatose, L-tagatose, or a mixture of the two isomers.
